(19) United States
(12) Patent Application Publication
(10) Pub. No.: US 2001/0039351 A1
(43) Pub. Date: Nov. 8, 2001

(54) Novel process

(75) Inventor: Stephen Ch. Eyley, Ashby de la Zouch (GB)

Correspondence Address: Nixon & Vanderhye P.C., 8th Floor, 1100 N. Glebe Rd., Arlington, VA, 22201, US

(73) Assignee: AstraZeneca AB

(21) Appl. No.: 09/842,710

(22) Filed: Apr. 27, 2001

Related U.S. Application Data
(62) Division of application No. 09/581,297, filed on Jun. 12, 2000.

(30) Foreign Application Priority Data
Feb. 26, 1999 (SE) 9900693-4
Feb. 22, 2000 (SE) PCT/SE00/00347

(51) Int. Cl. C07D 277/68

(52) U.S. Cl. 548/157

(57) ABSTRACT

The invention relates to a process for preparing benzothiazolone compounds having pharmacological activity and to intermediates used in their preparation.

The present invention relates to a process for preparing benzothiazolone compounds having pharmacological activity and to intermediates used in their preparation.

WO 93/24473 describes a class of benzothiazolone compounds, having both β₂-adrenoreceptor agonist activity and dopamine DA₂ receptor agonist activity, of general formula

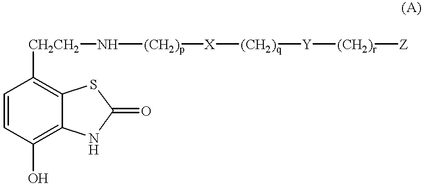

wherein X and Y independently represent —S(O)ₙ— or —O—,

n represents 0, 1 or 2,

p, q and r independently represent 2 or 3,

Z represents phenyl optionally substituted by halogen, OR¹, NO₂ or NR²R³; or a 5- or 6-membered N, O or S containing heterocycle, and

R¹, R² and R³ independently represent hydrogen or C₁-C₆ alkyl, and pharmaceutically acceptable derivatives thereof.

Example 6 of WO 93/24473 describes a compound of formula (A) in which X is SO₂, Y is O, p is 2, q is 3, r is 2 and Z represents a phenyl group. The compound of formula (A) is prepared by the selective reduction of a compound of formula (B)

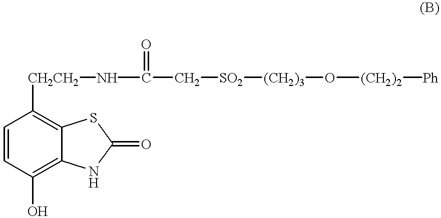

wherein Ph represents a phenyl group, in a borane-tetrahydrofuran solution.

The present invention provides an alternative process for preparing the compound of Example 6 of WO 93/24473 which avoids the need to use the intermediate of formula (B) and the potential hazards associated with using toxic and expensive borane reagents. Furthermore, the alternative process is simpler and more convenient to operate, resulting in good yields of crystalline product with minimal work-up.

In accordance with the present invention, there is therefore provided a process for the preparation of a compound of formula (I), or a pharmaceutically acceptable salt or solvate thereof,

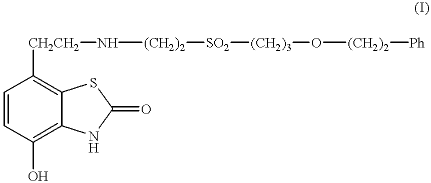

wherein Ph represents a phenyl group, which comprises reacting a compound of formula (II) or a salt thereof such as a hydrochloride or hydrobromide salt,

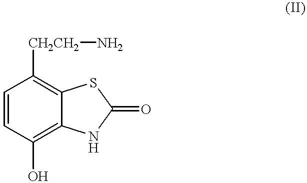

with a compound of formula (III),

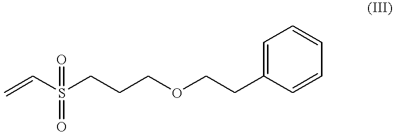

in the presence of a solvent and, optionally, a tertiary amine base; and, if desired, converting the compound of formula (I) to a pharmaceutically acceptable salt or solvate thereof.

In the present specification, unless otherwise indicated, an alkyl substituent group may be linear or branched. Further, the alkyl groups in a tri-C₁-C₆ alkylamine compound may be the same or different.

In the process of the invention, when using a salt of a compound of formula (II), a tertiary amine base will be present but when using a compound of formula (II), the base need not necessarily be present.

The tertiary amine base may be an aliphatic amine (e.g. tri-C₁-C₆ alkylamine such as triethylamine or N,N-diisopropylethylamine) or a heterocyclic amine comprising one or more fused rings and at least one ring nitrogen atom such as 1,8-diazabicyclo[5.4.0]undec-7-ene (DBU), 1,5-diazabicyclo[4.3.0]non-5-ene (DBN) or as 1,4-diazabicyclo[2.2.2]octane (DABCO). The tertiary amine base is conveniently used in an amount such that the molar ratio (mol/mol) of tertiary amine base to formula (II) compound/salt is in the range from 1 to 5, preferably in the range from 2 to 4.

The solvent used in the present process is preferably an organic solvent such as an alcohol, e.g. methanol or ethanol, or an amide such as dimethylformamide. The weight ratio (w/w) of solvent to formula (II) compound/salt is conveniently in the range from 5 to 30, preferably in the range from 5 to 25, and more preferably in the range from 5 to 20. The process of the present invention is preferably carried out at a temperature in the range from 15 to 100 ° C., more preferably from 50 to 100 ° C. and, in particular, at the reflux temperature of the solvent.

The pharmaceutically acceptable salts of the compound of formula (I) include acid addition salts derived from an inorganic or organic acid such as hydrochloric, hydrobromic, boric, phosphoric, sulfuric, acetic, tartaric, maleic, citric, succinic, ascorbic, benzoic, 4-methoxybenzoic, 2- or 4-hydroxybenzoic, 4-chlorobenzoic, benzenesulfonic, para-toluenesulfonic, naphthalenesulfonic, methanesulfonic, sulfamic, salicylic, diphenylacetic, triphenylacetic, adipic, fumaric, lactic, glutaric, gluconic, 1-hydroxy or 3-hydroxy-2-naphthoic or oleic acid. The compound of formula (I) and its pharmaceutically acceptable salts may also form pharmaceutically acceptable solvates such as hydrates.

The compound of formula (II) is a known compound which may be prepared, for example, as described by Weinstock et al., J. Med. Chem., 30, 1166-1176 (1987).

The compound of formula (III) is a novel compound and hence forms another aspect of the present invention.

In a preferred embodiment of the present process, the compound of formula (III) is formed in situ from a compound of general formula (IV),

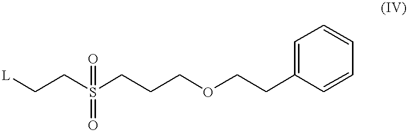

wherein L represents a carboxylate, e.g. benzoate, leaving group.

Compounds of formula (IV) are novel compounds and therefore form a further aspect of the present invention.

A compound of formula (IV) may conveniently be prepared by reacting a compound of formula (V),

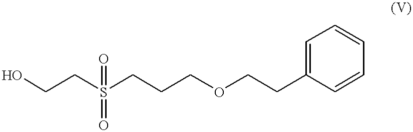

with a suitable acylating agent, e.g. an acid chloride such as benzoyl chloride. The reaction will typically be carried out in a solvent, e.g. a chlorinated solvent or an ester solvent such as ethyl acetate or isopropyl acetate, and a base such as triethylamine or sodium hydroxide.

The compound of formula (V) is a novel compound and forms another aspect of the present invention.

The compound of formula (V) may be readily prepared by contacting a compound of formula (VI),

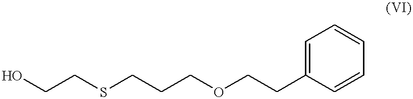

with an oxidising agent. Suitable oxidising agents to use include hydrogen peroxide, magnesium monoperoxyphthalate (MMPP), 3-chloroperoxybenzoic acid or potassium peroxymonosulphate, commercially sold under the trade mark “OXONE”. The reaction may conveniently be carried out in a solvent such as acetonitrile or dichloromethane, for example, at 0° C. to 70° C.

The compound of formula (VI) is a novel compound and forms a still further aspect of the present invention.

The compound of formula (VI) can be prepared by reacting a compound of formula (VII),

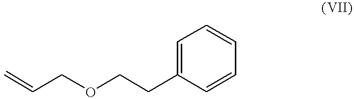

with 2-mercaptoethanol. The reaction is conveniently carried out in the presence of an initiator such as α,α′- azodiisobutyronitrile (AIBN) and a solvent such as toluene at a temperature in the range from 40 to 110 ° C.

The compound of formula (VII) is a known compound and may be prepared by techniques conventional in the art, for example, by reacting phenethyl alcohol with 3-bromopropene in the presence of a base such as sodium hydroxide and in a solvent such as toluene (see J. Amer. Chem. Soc. (1955), 77, 3889-3892).

The invention will now be further described by reference to the following illustrative Examples.

### EXAMPLE 1

### 4-Nitrobenzoic acid, 2-(3-(2-phenylethoxy)propylsulphonyl)ethyl ester

a) 2-(3-(2-Phenylethoxy)propylsulphonyl)ethanol

2-(3-(2-Phenylethoxy)propylsulphonyl)ethanol may be prepared from the 2-(2-propenyloxy)ethylbenzene (Cookson, R. C.; Wallis, S. R., J. Chem. Soc. B; 1966; 1245-1256) by the radical addition of 2-mercaptoethtanol, followed by oxidation using a hydrogen peroxide-based oxidant.

b) 4-Nitrobenzoic acid, 2-(3-(2-phenylethoxy)propylsulphonyl)ethyl ester

4-Nitrobenzoyl chloride (27.9 g, 0.15 mol) was dissolved in 2-propyl acetate (80 ml) and was added to a cooled, stirred solution of 2-(3-(2-phenylethoxy)propylsulphonyl)ethanol (34 g, 0.12 mol) and triethylamine (21 ml, 0.15 mol) in 2-propyl acetate (150 ml) at a rate to maintain the temperature below 25 ° C. (approx. 8 minutes). The mixture was stirred vigorously for a further two hours. A saturated solution of sodium hydrogen carbonate was then added, and the aqueous layer was removed. The organic layer was washed with water (2×50 ml) and then concentrated under reduced pressure to give the titled ester product as an off-white solid (42.5 g). The product was recrystallised from ethanol (400 ml) to give the ester as needles.

Melting point: 72-79 ° C.

¹H NMR (D₆-DMSO): δ 8.36, 8.15 (AA′BB′, 4H), 7.22 (m, 5H), 4.70 (t, 2H), 3.68 (t, 2H), 3.56 (t, 2H), 3.48 (t, 2H), 3.20 (dd, 2H), 2.76 (t, 2H), 1.93 (m, 2H)

### EXAMPLE 2

### 4-Methoxybenzoic acid, 2-(3-(2-phenylethoxy)propylsulphonyl)ethyl ester

4-Methoxybenzoyl chloride (25.6 g. 0.15 mol) was dissolved in 2-propyl acetate (50 ml) and was added to a cooled stirred solution of 2-(3-(2-phenylethoxy)propylsulphonyl)-ethanol (34 g, 0.12 mol) and triethylamine (21 ml, 0.15 mol) in 2-propyl acetate (200 ml). The mixture was vigorously stirred for two hours. A saturated solution of sodium hydrogen carbonate (300 ml) was added, the organic layer was separated and washed with water (2×50 ml) and then concentrated under reduced pressure to give the titled ester product, which was purified by chromatography (over silica using mixtures of ethyl acetate/petroleum ether) to give the ester as a clear mobile oil.

MS 407 (M+H)⁺

¹H NMR (D₆-DMSO): δ 7.93, 7.05 (AA′BB′, 4H), 7.22 (m, 5H), 4.60 (t, 2H), 3.63 (t, 2H), 3.55 (t, 2H), 3.47 (t, 2H), 3.17 (m, 2H), 2.75 (t, 2H), 1.91 (m, 2H)

### EXAMPLE 3

### Benzoic acid, 2-(3-(2-phenylethoxy)propylsulphonyl)ethyl ester

2-(3-(2-Phenylethoxy)propylsulphonyl)ethanol (60 g, 0.22 mol) was dissolved in dichloromethane (400 ml) and benzoyl chloride (30.7 ml, 0.265 mol) was added in one portion. With stirring under nitrogen, triethylamine (36.8 ml) was added over 12 minutes, the temperature rising to 41 ° C. The resulting suspension was stirred for 20 hours, washed sequentially with water (100 ml) and saturated sodium hydrogen carbonate (1100 ml) and then dried over sodium sulphate. Filtration and concentration afforded an oil which readily crystallised. The titled product was purified by recrystallisation from ethanol to give needles (60.4 g, 72% yield).

Melting point: 65.5 ° C.

¹H NMR (CDCl₃): δ 8.03 (d, 2H), 7.60 (t, 1H), 7.45 (t, 2H), 7.25 (m, 4H), 4.74 (t, 2H), 3.60 (t, 2H), 3.51 (t, 2H), 3.38 (t, 2H), 3.10 (m, 2H), 2.81 (t, 2H), 2.09 (m, 2H)

### EXAMPLE 4

### 4-Hydroxy-7-(2-(2-(3-(2-phenylethoxy)propylsulphonyl)ethylamino)ethyl)-1.3- benzothiazol-2(3H)-one, hydrochloride

a) 2-(3-Ethenylsulphonylpropoxy)ethylbenzene

Benzoic acid, 2-(3-(2-phenylethoxy)propylsulphonyl)ethyl ester, (Example 3) (500 g, 1.33 mol) was dissolved in ethyl acetate (3.5 L) under nitrogen. 1,8-Diazabicyclo[5,4,0]-undec-7-ene (212.8 ml, 1.39 mol) was added and the mixture was stirred at ambient temperature for four hours. The precipitated solid was isolated by filtration, washed with ethyl acetate (1.5 L), and the combined organic phases washed with dilute hydrochloric acid (2×1 L), saturated sodium carbonate solution (2×1 L), and brine (1 L). The organic phase was dried over magnesium carbonate, and filtered and concentrated under reduced pressure to give the titled product as an oil (329 g, 97% yield) which crystallised on standing.

Melting point: 28-28.5 ° C.

MS (FAB) 255 (M+H)⁺

¹H NMR (D₆-DMSO): δ 7.25 (m, 5H), 6.97 (dd, 1H), 6.23 (m, 2H), 3.57 (t, 2H), 3.46 (t, 2H), 3.08 (t, 2H), 2.80 (t, 2H), 1.80 (m, 2H)

b) 4-Hydroxy-7-(2-(2-(3-(2-phenylethoxy)propylsulphonyl)ethylamino)ethyl)-1,3-benzothiazol-2(3H)-one, hydrochloride

7-(2-Aminoethyl)-4-hydroxy-1,3-benzothiazol-2(3H)-one, hydrochloride (100 g) was suspended in methanol (480 ml) at the reflux point. A solution of the 2-(3-ethenylsulphonylpropoxy)ethylbenzene (107 g) and triethylamine (53.8 ml) in methanol (240 ml) was added to the refluxing mixture over 35 minutes. After 2.75 hours, methanol (960 ml) was added, followed by hydrochloric acid (37 ml), and the mixture allowed to cool. Filtration, washing with propan-2-ol (400 ml), and then ether (400 ml), and drying afforded the hydrochloride salt (135.7 g, 69% yield).

MS 465 (M+H)⁺

¹H NMR (500 MHz spectrum) (D₆-DMSO): δ 11.75 (s, 1H), 10.12 (s, 1H), 9.42 (s, 2H), 7.17-7.30 (m, 5H), 6.88, 6.78 (ABq, 2H), 3.60 (t, 2×2H), 3.50 (t, 2H), 3.36 (t, 2H), 3.21 (m, 2H), 3.17 (t, 2H), 2.89 (t, 2H), 2.81 (t, 2H), 1.92 (m, 2H)

### EXAMPLE 5

### 4-Hydroxy-7-(2-(2-(3-(2-phenylethoxy)propylsulphonyl)ethylamino)ethyl)-1,3- benzothiazol-2(3H)-one, hydrochloride

7-(2-Aminoethyl)-4-hydroxy-1,3-benzothiazol-2(3H)-one, hydrochloride (1.0 g), benzoic acid, 2-(3-(2-phenylethoxy)propylsulphonyl)ethyl ester (Example 3) (1.52 g), triethylamine (2.26 ml), and methanol (20 ml) were heated to reflux temperature over 30 minutes. This temperature was maintained for 5.5 hours, by which time high pressure liquid chromatography indicated that the reaction was complete. The mixture was allowed to cool slightly, and then acidified by the addition of concentrated hydrochloric acid (1.71 ml). On cooling and stirring, a salt crystallised from solution. Isolation by filtration, washing with propan-2-ol, and drying gave the titled product (1.1 g, 54% yield).

### EXAMPLE 6

### 4-Hydroxy-7-(2-(2-(3-(2-phenylethoxy)propylsulphonyl)ethylamino)ethyl)-1,3- benzothiazol-2(3H)-one, hydrochloride

7-(2-Aminoethyl)-4-hydroxy-1,3-benzothiazol-2(3H)-one, hydrochloride (3.0 g), 4-nitrobenzoic acid, 2-(3-(2-phenylethoxy)propylsulphonyl)ethyl ester (Example 1) (5.7 g), triethylamine (5.96 ml), and ethanol (industrial methylated spirits, 60 ml) were heated to reflux temperature over 20 minutes. This temperature was maintained for 4 hours. The mixture was allowed to cool slightly, and then acidified by the addition of concentrated hydrochloric acid (4.6 ml). On cooling, a salt crystallised from solution. Isolation by filtration, washing with propan-2-ol, and drying gave the titled product (3.8 g 62% yield)

### EXAMPLE 7

### 4-Hydroxy-7-(2-(2-(3-(2-phenylethoxy)propylsulphonyl)ethylamino)ethyl)-1,3- benzothiazol-2(3H)-one, hydrochloride

The method described in Example 6 was repeated using the compound of Example 2 in place of the compound of Example 1. The titled product was obtained in 61% yield.

1. A process for the preparation of a compound of formula (I), or a pharmaceutically acceptable salt or solvate thereof, 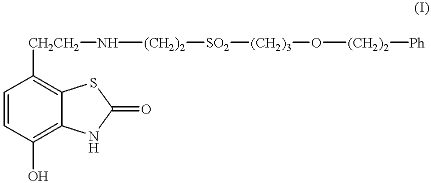
wherein Ph represents a phenyl group, which comprises reacting a compound of formula (II) or a salt thereof, 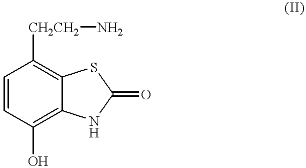
with a compound of formula (III), 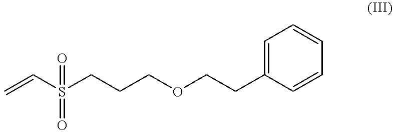
in the presence of a solvent and, optionally, a tertiary amine base; and, if desired, converting the compound of formula (I) to a pharmaceutically acceptable salt or solvate thereof.

2. A process according to claim 1 wherein the tertiary amine base is an aliphatic amine.

3. A process according to claim 1 or claim 2 wherein the solvent is an alcohol.

4. A process according to any one of claims 1 to 3, wherein the pharmaceutically acceptable salt is an acid addition salt derived from hydrochloric, hydrobromic, boric, phosphoric, sulfuric, acetic, tartaric, maleic, citric, succinic, ascorbic, benzoic, 4-methoxybenzoic, 2- or 4-hydroxybenzoic, 4-chlorobenzoic, benzenesulfonic, para-toluenesulfonic, naphthalenesulfonic, methanesulfonic, sulfamic, salicylic, diphenylacetic, triphenylacetic, adipic, fumaric, lactic, glutaric, gluconic, 1-hydroxy or 3-hydroxy-2-naphthoic or oleic acid.

5. A process according to any one of claims 1 to 4, wherein the compound of formula (III) is formed in situ.

6. A process according to claim 5 wherein the compound of formula (III) is formed in situ from a compound of general formula (IV), 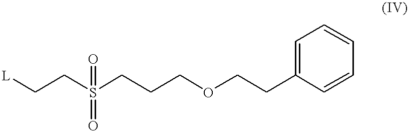
wherein L represents a carboxylate leaving group.

7. A compound of formula (III) as defined in claim 1.

8. A compound of formula (IV) as defined in claim 6.

9. A process for preparing a compound of formula (IV) as defined in claim 6 which comprises reacting a compound of formula (V), 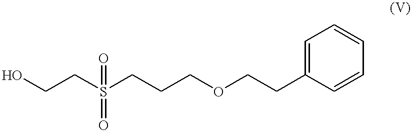
with a suitable acylating agent.

10. A process according to claim 9, wherein the compound of formula (V) is prepared by contacting a compound of formula (VI), 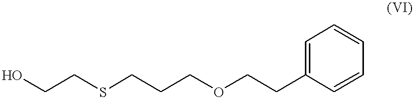
with an oxidising agent.

11. A process according to claim 10, wherein the compound of formula (VI) is prepared by reacting a compound of formula (VII), 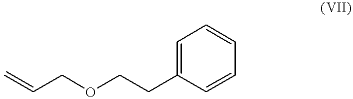
with 2-mercaptoethanol.

12. A process for preparing a compound of formula (IV) as defined in claim 6 which comprises:
(a) reacting phenethyl alcohol with 3-bromopropene to obtain a compound of formula (VII), 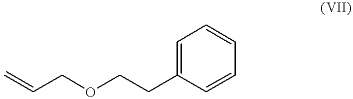
(b) reacting the compound of formula (VII) with 2-mercaptoethanol to obtain a compound of formula (VI), 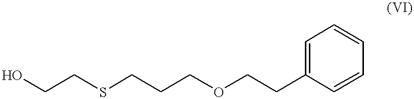
(c) oxidising the compound of formula (VI) to obtain a compound of formula (V), 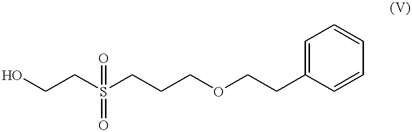
and
(d) reacting the compound of formula (V) with a suitable acylating agent to obtain a compound of formula (IV).

13. A compound of formula (V) as defined in claim 9 or claim 12.

14. A compound of formula (VI) as defined in claim 10 or claim 12.
